(19) United States
(12) Patent Application Publication
(10) Pub. No.: US 2023/0057938 A1
(43) Pub. Date: Feb. 23, 2023

(54) DISPLAY OF FITNESS ACTIVITY DATA ON DISPLAY DEVICE

(71) Applicant: ARRIS Enterprises LLC, Suwanee, GA (US)

(72) Inventor: Vijaykumar KHANVILKAR, Bangalore (IN)

(21) Appl. No.: 17/721,552

(22) Filed: Apr. 15, 2022

Related U.S. Application Data
(60) Provisional application No. 63/234,361, filed on Aug. 18, 2021.

(51) Int. Cl. G16H 40/67; G16H 20/30; G16H 20/60

(52) U.S. Cl. CPC G16H 40/67; G16H 20/30; G16H 20/60

(57) ABSTRACT

A content output device includes a memory, and a processor configured to execute the computer-readable instructions to receive a fitness activity trigger event associated with at least one registered user, launch a fitness activity display application, retrieve fitness activity data of at least one user, and display, on a display device, a fitness activity report associated with the fitness activity of the user. The fitness activity data is obtained by a fitness activity tracker device or a mobile device, or from a remote server. Fitness activity data may be provided to the fitness activity display application. The fitness activity trigger event is generated with a designated key on a remote control is selected. The fitness activity display application processes the fitness activity data to generate the fitness activity report for presentation on a display device.

### BACKGROUND

The subject matter of the present disclosure relates generally to displaying fitness activity data on a display device, such as a television.

Fitness activity trackers are popular digital gadgets that track various fitness activities and provide health related statistical data useful for the users to achieve fitness and/or medical goals. They may be fitness bands or smartwatches consisting of various features: steps tracking, heart rate tracking, sleeping pattern tracking, active time tracking, exercise routines, stress levels, calories burnt, health-related measurements, etc.

Fitness activity trackers are also capable of sending emergency alerts in case of any activity reading detected above threshold like higher heart rate. Such devices also provide various exercise routines to achieve required fitness goals. The fitness activity data is viewable directly on a tracker device or through application provided by device manufacturer or some third-party application installed on a phone that is paired with a fitness activity tracker. These activity trackers consist of various sensors for tracking activities and for synchronizing data to a mobile device. Nowadays, even mobile devices consist of similar sensors for tracking activities in which case an additional tracker device is not necessary. The fitness activity trackers provide useful statistical data about activities and health to help attain a user's health and fitness goals. Therefore, fitness activity data should be easily accessible to user.

### SUMMARY

An aspect of the present disclosure involves a system and method to initiate the display of fitness activity data on a display device, such as a television.

A content output device includes a memory storing computer-readable instructions, and a processor configured to execute the computer-readable instructions to receive fitness activity trigger event associated with at least one registered user, launch fitness activity display application, retrieve fitness activity data associated with the at least one registered user, and display, on a display device, a fitness activity report associated with the fitness activity of the at least one registered user. The fitness activity data is obtained by a fitness activity tracker device of a registered user and the fitness activity data associated with the registered user is provided directly to the fitness activity display application. The fitness activity data is obtained by a fitness activity tracker device of the registered user and the fitness activity data associated with the at least one registered user is stored in a server.

The fitness activity tracker device of the registered user provides the fitness activity data associated with the at least one registered user to a mobile phone, and wherein the mobile phones sends the fitness activity data associated with the at least one registered user to the server, which may be a local server or a remote cloud server. The fitness activity tracker device of the registered user provides the fitness activity data associated with the at least one registered user to the server. The fitness activity trigger event associated with the registered user is provided by a remote control device having a designated key that when selected generates the fitness activity trigger event that is received. The fitness activity display application includes a graphical user interface for receiving user input to control presentation of the fitness activity report, and to control registration and de-registration of an account associated with at least one registered user, wherein the fitness activity display application processes the fitness activity data associated with the registered user according to the user input to generate the fitness activity report for display. The fitness activity data may be associated with two or more registered users, and the fitness activity display application presents one or more of a comprehensive report comparing the fitness activity data of the two or more registered users, a recommendation of at least one of exercise and diet, and fitness goals suggested for the two or more registered users.

### BRIEF SUMMARY OF THE DRAWINGS

The accompanying drawings, which are incorporated in and form a part of the specification, illustrate examples of the subject matter of the present disclosure and, together with the description, serve to explain the principles of the present disclosure. In the drawings:

FIG. 1 illustrates a user exercising.

FIG. 2 illustrates the transfer of fitness activity data.

FIG. 3 illustrates a fitness key on a remote control.

FIG. 4 illustrates operation of the fitness key on a remote control.

FIG. 5 illustrates operation of a content delivery device.

FIG. 6 illustrates a GUI displaying tracker data.

FIG. 7 is a flow chart of a method for displaying fitness activity data on a display device.

FIG. 8 illustrates a block diagram of a device, such as a fitness activity tracker device, mobile device, and/or a content delivery device as discussed above.

### DETAILED DESCRIPTION

The following detailed description is made with reference to the accompanying drawings and is provided to assist in a comprehensive understanding of various example embodiments of the present disclosure. The following description includes various details to assist in that understanding, but these are to be regarded merely as examples and not for the purpose of limiting the present disclosure as defined by the appended claims and their equivalents. The words and phrases used in the following description are merely used to enable a clear and consistent understanding of the present disclosure. In addition, descriptions of well-known structures, functions, and configurations may have been omitted for clarity and conciseness.

Aspects of the present disclosure are directed to a system and method to initiate the display of fitness activity data on a display device, such as a television.

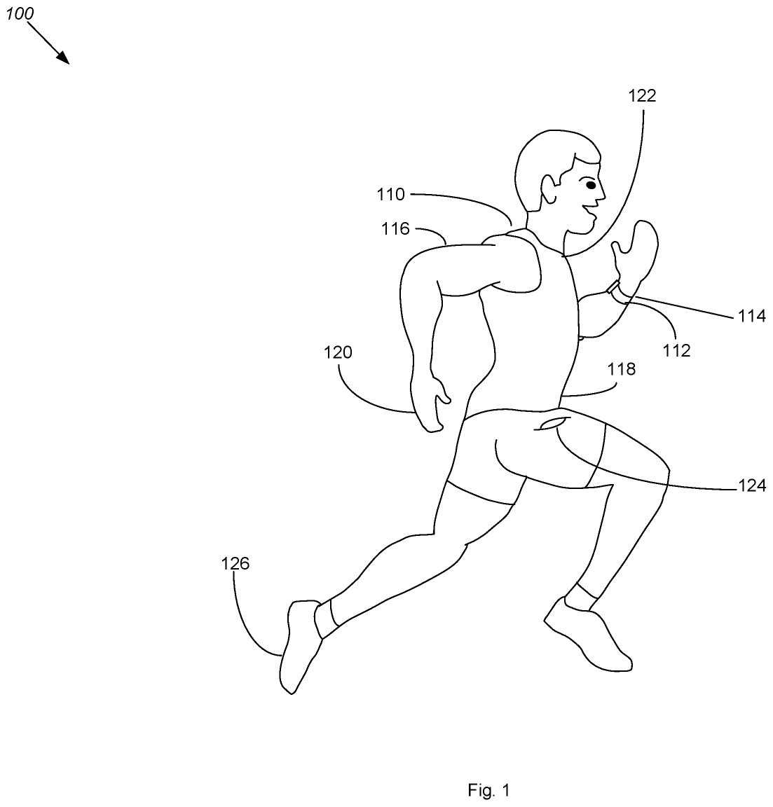

FIG. 1 illustrates a user exercising 100.

FIG. 1 shows a user 110 is shown exercising while wearing a fitness activity tracker 112. In FIG. 1, the fitness activity tracker 112 is illustrated as a watch or other device worn on the wrist 114 of the user 110. However, the fitness activity tracker 112 could be a ring worn on the finger 120 of the user 110, an application that runs on a mobile phone or other device worn on the upper arm 116 or around the user's waist 118, a clip-on device worn at the waist or clipped to the shirt 122 or a pocket 124, an insert that can be worn inside a user's shoe 126, etc. Fitness activity tracker 112 collects fitness activity data of the user. Fitness activity data may include number of steps taken by a user, heart rate data, sleeping patterns, active time tracking, exercise routines, stress levels, calories burnt, blood pressure, glucose measurement, suggestions, etc. The collected fitness activity data may be used to provide useful statistical data about a user's activities and health to help attain health and fitness goals.

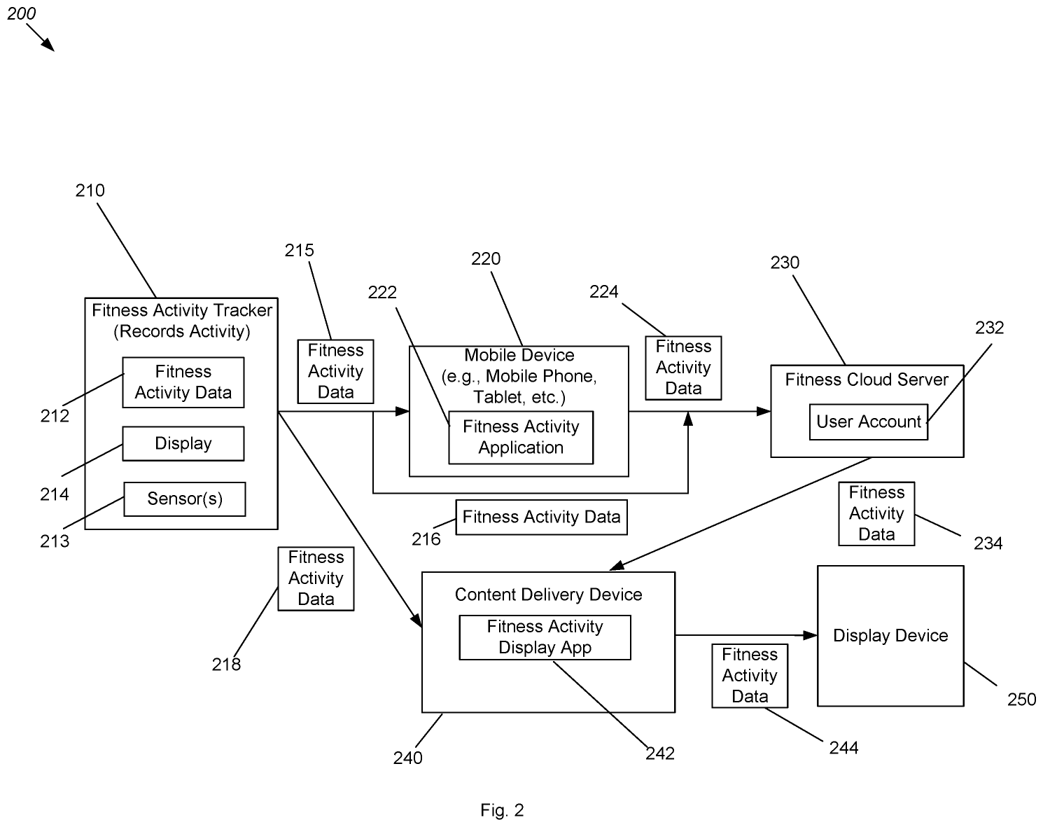

FIG. 2 illustrates the transfer of fitness activity data 200.

FIG. 2 shows a fitness activity tracker 210 that records fitness activity data 212, e.g., in real time. There may multiple fitness activity trackers 210 for multiple users. Fitness activity tracker 210 may include one or more sensors 213 for gathering fitness activity data 212. The fitness activity data 212 obtained by the one or more sensors 213 may displayed to a user of the fitness activity tracker on a display 214. The fitness activity tracker 210 may be paired with a mobile device 220, such as a mobile phone, tablet, etc., that runs a fitness activity application 222. For example, the fitness activity tracker 210 may be paired with the mobile device 220 using a wireless connection, such as a Wi-Fi connection, Bluetooth connection, etc. Fitness activity tracker 210 triggers an event to fitness activity application 222 on mobile device 220 to cause the fitness activity application 222 to read the fitness activity data 215. The fitness active data 215 may thus be transferred to the fitness activity application 222 on mobile device 220. The fitness activity application 222 may display on the mobile device 220 the fitness activity data 214 or a fitness activity report based on fitness activity data 215.

The fitness activity application 220 may then transfer the fitness activity data 224 to a user associated account 232 at a server, such as fitness cloud server 230. The fitness activity tracker 210 may transfer the fitness activity data 216 directly to user account 232 at fitness cloud server 230 rather than to the fitness activity application 222 on mobile device 220. The fitness cloud server 230 may transfer the fitness activity data 234 to a content delivery device 240. The fitness activity tracker 210 may transfer the fitness activity data 218 directly to the content delivery device 240 rather than to the fitness activity application 222 on mobile device 220 or to user account 232 at the fitness cloud server 230. The content delivery device 240 executes a fitness activity display application 242 to process fitness activity data and send information associated with the fitness activity data 244 for presentation in the foreground on display device 250, such the fitness activity report. The application 242 may be a native application of the content delivery device 240, or may otherwise be a third-party fitness application.

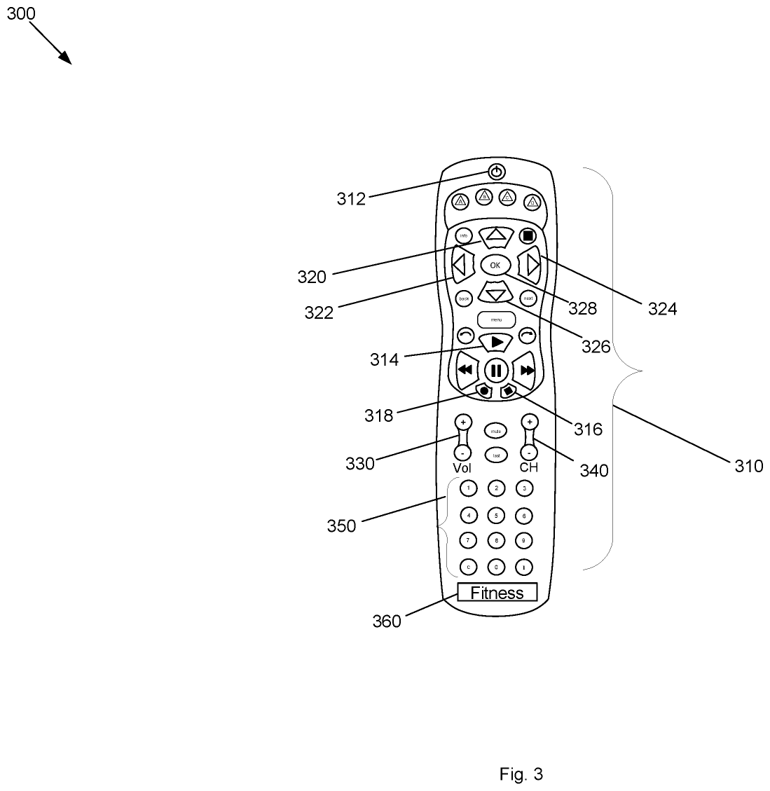

Fitness activity display application 242 processes the fitness activity data 218 and/or fitness activity data 234 to generate useful statistical data about a user's activities and health to help attain health and fitness goals. Fitness activity display application 242 may also provide consolidated fitness activity data about multiple users, e.g., gather by multiple fitness activity trackers 210. Therefore, it is important that the fitness activity data should be easily accessible to the user. Fitness activity display application 242 provides such data for presentation on the display device 250, such as a television. The fitness activity display application 242 may be linked to the fitness key on a remote control and may be launched when the fitness key is pressed, as will be explained in greater detail next with regard to FIG. 3.

FIG. 3 illustrates a fitness key on a remote control 300.

In FIG. 3, remote control 300 includes buttons 310 for controlling a content delivery device, such as a smart home device or set-top box. For example, FIG. 3 shows buttons for power 312, play 314, stop 316, record 318, up navigation 320, left navigation 322, right navigation 324, down navigation 326, select 328, volume control 330, channel control 340, and a numerical keypad 350. Other control buttons may also be provided, but will not be described herein.

FIG. 3 also shows a special functional key, referred to herein as a fitness key 360. In FIG. 3, the fitness key 360 is labeled FITNESS, on the remote control 300. The fitness key 360 may be mapped to perform any action related to fitness or health activity. However, fitness key 360 herein is pressed to generate a fitness activity trigger event that is sent to the content control device. Thus, fitness key that is used to trigger display of fitness activity data on a display device through a content delivery device, such as smart home device or set-top box. The remote control 300 with the fitness key 360 may communicate with a content delivery device by transmitting IR or Bluetooth signals. Fitness activity data may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc.

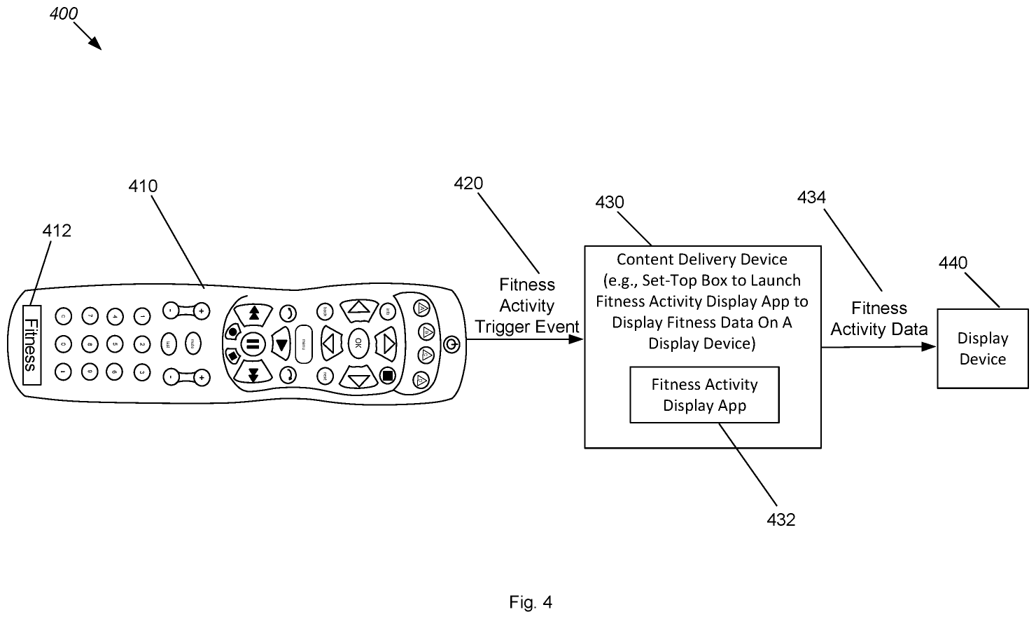

FIG. 4 illustrates operation of the fitness key on a remote control 400.

In FIG. 4, a fitness key 412 on a remote control 410 may be pressed to generate a fitness activity trigger event 420. The fitness activity trigger event 420 is sent to a content delivery device 430, e.g., a set-top box. Upon receiving the fitness activity trigger event 420, a fitness activity display application 432 is launched on the content delivery device 430. The fitness activity display application 432 sends fitness activity data 434 to a display device 440. Fitness activity data 434 may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc. When a fitness key 412 is not available on a remote control 410, an alternative method of sending a fitness activity trigger event 420—may be provided. For example, a user may use the remote control 410 or other input to the content delivery device 430 to browse a user interface (UI) to select and launch the fitness activity display application 432.

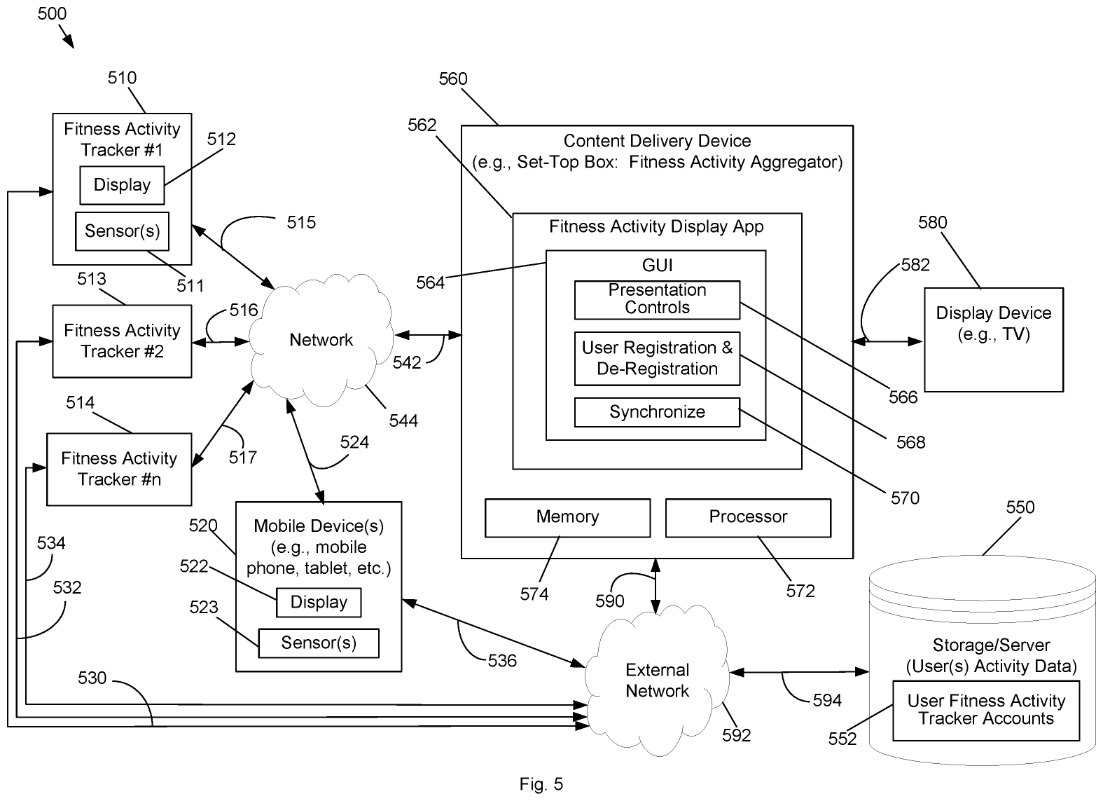

FIG. 5 illustrates operation of a content delivery device 500.

In FIG. 5, fitness activity trackers #1 510, #2 513, #n 514 are coupled to a network 544. A mobile device, such as a mobile phone, tablet, etc., and content delivery device 560 may also connect to network 544. The connections 515, 516, 517, 524, 540, 542 between network 544 and fitness activity trackers #1 510, #2 513, #n 514, mobile device(s) 520, and content delivery device 560 can be implemented using a wireless connection in accordance with any IEEE 802.11 Wi-Fi protocols, Bluetooth protocols, Bluetooth Low Energy (BLE), or other short range protocols that operate in accordance with a wireless technology standard for exchanging data over short distances using any licensed or unlicensed band such as the citizens broadband radio service (CBRS) band, 2.4 GHz bands, 5 GHz bands, 6 GHz bands, 60 GHz bands, etc. Additionally, the connections 515, 516, 517, 524, 540, 542 can be implemented using a wireless connection that operates in accordance with, but is not limited to, Radio Frequency for Consumer Electronics (RF4CE) protocol, ZigBee protocol, Z-Wave protocol, or IEEE 802.15.4 protocol. It is also contemplated by the present disclosure that the connection 109 can include connections to a media over coax (MoCA) network. One or more of the connections 109 can also be a wired Ethernet connection.

Content delivery device 560 may access remote server 550 through external network 592 using connections 590, 594. Fitness activity trackers #1 510, #2 513, #n 514 and mobile device 520 may also access remote server 550 through external network 592 using connections 530, 532, 534, 536. Connections 530, 532, 534, 536, 590, 594 are capable of providing connections to remote server 550 through WAN, a LAN, a VPN, MANs, personal area networks (PANs), wireless LANs (WLANs), SANs, a DOCSIS network, a fiber optics network (e.g., FTTH, FTTX, or HFC), a PSDN, a global Telex network, or a 2G, 3G, 4G or 5G network, for example.

Fitness activity trackers #1 510, #2 513, #n 514 gather data from various sensors and may display such information thereon. Further, fitness activity data from fitness activity trackers #1 510, #2 513, #n 514 may be associated with multiple users of fitness activity trackers #1 510, #2 513, #n 514, e.g., such as members of a family. Fitness activity trackers #1 510, #2 513, #n 514 may send fitness activity data to mobile device 520 using network 544. As discussed with regard to FIG. 2, fitness activity trackers #1 510, #2 513, #n 514 may also, or alternatively, send fitness activity data directly to a server 550 (e.g., via a local or a cloud server) and/or to content delivery device 560. Server 550 may store fitness activity data from fitness activity trackers #1 510, #2 513, #n 514 into a database periodically. Fitness activity data may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc.

When the mobile device 520 receives the fitness activity data from fitness activity trackers #1 510, #2 513, #n 514, mobile device 520 may send the fitness activity data to server 550 via network 540. In another embodiment, fitness activity trackers #1 510, #2 513, #n 514 may communicate directly with the content delivery device 560. Content delivery device 560 is coupled to a display device 580, such as a television. Content delivery device 560 may be a smart home device, set-top box, or other device that is capable of delivering content to the display device 580. Connection 582 between content delivery device 560 and display device 580 may be any analog or digital connection, such as High-Definition Multimedia Interface (HDMI), coaxial, fiber optic, Radio Corporation of America (RCA) composite, Super (S)-video, Universal Serial Bus (USB), etc.

Content delivery device 560 may further retrieve fitness activity data associated with fitness activity trackers #1 510, #2 513, #n 514 from server 550 through network 540 or via external network 590, such as the Internet. Connection 592 between external network 590 and content delivery device 560, connection 594 between external network 590 and server 550 may be a wide area network (WAN) connection, a virtual private network (VPN) connection, metropolitan area networks (MANs) connection, a DOC SIS (Data Over Cable Service Interface Specification) network connection, a fiber optics network connection (e.g., FTTH (fiber to the home) or FTTX (fiber to the x), or hybrid fiber-coaxial (HFC)), a digital subscriber line (DSL) connection, a public switched data network (PSDN) connection, a global Telex network connection, or a 2G, 3G, 4G or 5G network connection, for example.

Content delivery device 560 implements a fitness activity display application 562. Fitness activity display application 562 may process fitness activity data and present such data using a graphical user interface (GUI) 564 that is displayed on display device 580. Content delivery device 560 includes instructions in memory 574 that, when executed by processor 572, may retrieve fitness activity data from server 550. Content delivery device 560 may then process the fitness activity data and send a fitness report to display device 580. As mentioned earlier, another approach may be used where fitness activity data is directly received from a fitness application of mobile device(s) 520, or from fitness activity trackers #1 510, #2 513, #n 514,

The content delivery device 560 creates a fitness activity report based on the received fitness activity data that is presented on display device 580. Based on the fitness activity data, emergency alerts may also be generated by the fitness activity display application 562 to provide a warning or other message on the display device 580 in case activity readings are above threshold. Display device 580 presents information associated with the fitness activity data from fitness activity trackers #1 510, #2 513, #n 514 in the foreground on a display of display device 580. There are no sensors directly connected to the content delivery device 560 or the display device 580 to provide fitness activity data. The content delivery device 560 and the display device 580 act as an aggregator for retrieving and displaying fitness activity data gathered by fitness activity trackers #1 510, #2 513, #n 514. The fitness activity data gathered by one or more sensors 511 is viewable directly on a display 512 of fitness activity trackers #1 510, #2 513, #n 514, or through a fitness tracking application 522 provided by device manufacturer or some third-party fitness tracking application installed on a mobile device 520 that is paired with the fitness activity trackers #1 510, #2 513, #n 514.

Fitness activity trackers #1 510, #2 513, #n 514 include various sensors 511 for tracking fitness activities and synchronize fitness activity data to mobile device(s) 520, server 550, and/or content delivery device 560. There may be a mobile device 520 associated with each fitness activity tracker #1 510, #2 513, #n 514. The one or more mobile devices 520 may also include similar sensors 523 for tracking fitness activities in which case an additional or separate fitness activity trackers #1 510, #2 513, #n 514 are not necessary.

GUI 564 provided by fitness activity display application 562 executed by the content delivery device 560 provides presentation controls 566 to manage how information associated with fitness activity data is displayed. The graphical user interface 564 also provides controls for user registration and de-registration 568 so user fitness activity tracker accounts 552 may be created at server 550.

The fitness activity display application 562 runs in the background to retrieve fitness activity data periodically from fitness activity trackers of all registered users (directly from fitness activity trackers #1 510, #2 513, #n 514, mobile device 520, or server 550). Retrieved fitness activity data may be stored in memory 574 for processing by fitness activity display application 562 under control of processor 572.

Fitness activity trackers #1 510, #2 513, #n 514 may be used to provide input to the user registration and de-registration controls 568 using menu options presented by the GUI 564. The user registration and de-registration controls 568 allow the users of fitness activity trackers #1 510, #2 512, #n 514 to dynamically add, delete, or otherwise control user fitness activity tracker accounts 552 on server 550. If a fitness activity tracker is de-registered, then data is not read for it from corresponding platform interface. A synchronic control 570 is provided in GUI 564 for synchronizing fitness activity data.

As described above, user fitness activity tracker accounts 552 for registered users may be created on a server 550 using the user registration and de-registration controls 568. Each user fitness activity tracker account 552 is linked to one of fitness activity trackers #1 510, #2 513, #n 514, and/or one or more mobile devices 520 for storing fitness activity data associated with a user of activity trackers #1 510, #2 513, #n 514 and/or a mobile device 520. The fitness activity data is accessible by a user to view or modify. For example, if a user is associated with fitness activity tracker #1, a user fitness activity account 552 is created on the server 550 where all fitness activity data associated with fitness activity tracker #1 is stored. Other accounts, fitness activity trackers and/or mobile devices, and associated fitness activity data operate similarly. The fitness activity display application 562 and server 550 also provide a way to access fitness activity data for other third-party applications through defined set of APIs.

The account credentials, a make of one or more fitness activity trackers #1 510, #2 513, #n 514 and/or one or more mobile device 520, as well as other basic user information, is securely stored in memory 574 of the content delivery device 560 and/or the server 550. The account credentials are used to access user fitness activity tracker accounts 552, to read fitness activity data from the server 550, or to read fitness activity data directly from the fitness activity trackers #1 510, #2 513, #n 514 or mobile device 520.

When a fitness key is pressed on a remote control, a fitness activity trigger event is sent to the content control device 560. The fitness activity display application 562 is launched and is used to retrieve fitness activity data from associated user fitness activity tracker accounts 552 of one or more users.

The fitness activity display application 562 running on the content delivery device 560 accesses fitness activity data from one or more user based on registered user fitness activity tracker accounts 552. Fitness activity display application 562 generates a graphical user interface (GUI) 564 to present on display device 580. The fitness activity display application 562 may be a native application of the content delivery device 560, or may otherwise be a third-party fitness application. The fitness activity display application 562 may be linked to the fitness key and launched when the fitness key is pressed.

The fitness activity data from fitness activity trackers #1 510, #2 513, #n 514 and/or mobile device(s) 520 is consolidated by the fitness activity display app 562 of content delivery device 560 and then fitness activity data for all users is sent for presentation on display device 580. Selection of Synchronic control 570 provides consolidation of fitness activity data from fitness activity trackers #1 510, #2 513, #n 514 and/or mobile device(s) 520.

Fitness activity display application 562 of content delivery device 560 may thus generate a comprehensive report, graphical comparison, provide recommendations etc. by using the fitness activity data from fitness activity trackers #1 510, #2 513, #n 514 and/or mobile devices(s) 520 associated with multiple users. When a user requests for a fitness report by pressing the fitness key on a remote control, the fitness activity display application 562 prepares a consolidated report for all registered user fitness activity tracker accounts 552 to display on the display device 580. The fitness activity display application 562 may also learn about the data to present various comparison, recommendation of exercise or diet, animation, and various other reports to help any individual user or family or friends to plan and achieve fitness goals.

Access to user fitness activity tracker accounts 552 may be controlled according to rules defined by a fitness activity tracker manufacturer. For example, FitBit® trackers provides Web APIs to access user activity data through HTTP requests using a FitBit® tracker account as per its defined rules. This allows a registered user to access or modify the user's fitness activity data, including use of third-party applications, wherein the fitness activity display application 562 retrieves fitness activity data on behalf of user.

There are many other fitness activity trackers #1 510, #2 513, #n 514 like Apple's HealthKit® application, Google Fit® application, etc., that provide a similar platform. As of now, there are no standard defined on data accessibility from so called health devices, e.g., fitness activity trackers fitness activity trackers #1 510, #2 513, #n 514. If such standard is once defined and supported by fitness activity tracker manufacturers, an easier solution may be provided to access fitness activity data through defined APIs across fitness activity trackers fitness activity trackers #1 510, #2 513, #n 514. Currently, there is no generic APIs for data accessibility to access fitness activity data across fitness activity trackers fitness activity trackers #1 510, #2 513, #n 514, so a platform specific implementation is required. This is possible by publishing supported platforms based on the make of the fitness activity trackers #1 510, #2 513, #n 514.

The fitness activity display application 562 uses settings provided by a user about a make of the fitness activity trackers #1 510, #2 513, #n 514 of the user and then maps it to a corresponding known platform for data accessibility. The fitness activity display application 562 will then invoke corresponding APIs of the supported platform to read fitness activity data from corresponding fitness activity trackers #1 510, #2 513, #n 514, mobile device(s) 520, and/or server 550. Fitness activity trackers #1 510, #2 513, #n 514 may not be supported if no such platform is supported by fitness activity trackers #1 510, #2 513, #n 514.

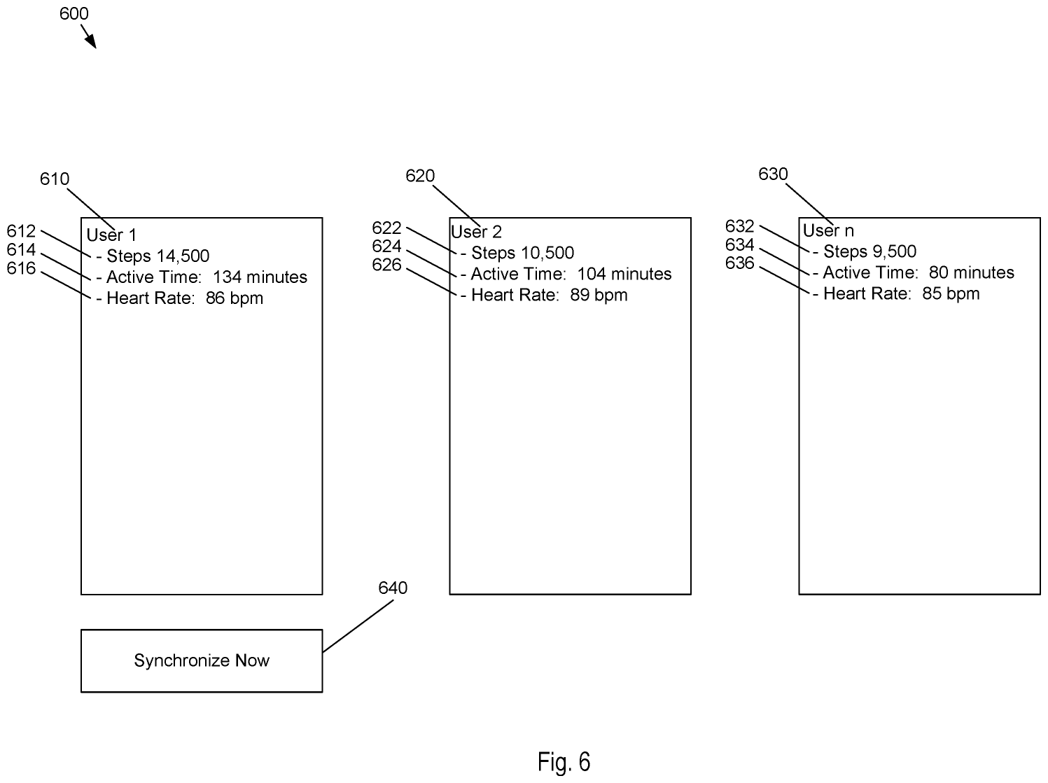

FIG. 6 illustrates a GUI 600 displaying tracker data.

In FIG. 6, tracker data for user 1 610 is display. Tracker data for user 1 610 shows that user 1 has taken 14,500 steps 612, an activity time of 134 minutes 614, and a heart rate of 86 bpm 616. Tracker data for user 2 620 shows that user 2 has taken 10,500 steps 622, an activity time of 104 minutes 624, and a heart rate of 89 bpm 626. Tracker data for user 3 630 shows that user n has taken 9,500 steps 632, an activity time of 80 minutes 634, and a heart rate of 85 bpm 636. The GUI 600 includes a synchronize now button 640 is provided for synchronizing data. Selection of the synchronize now button 640 provides consolidation of fitness activity data from fitness activity trackers and/or mobile device(s). Fitness activity data may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc.

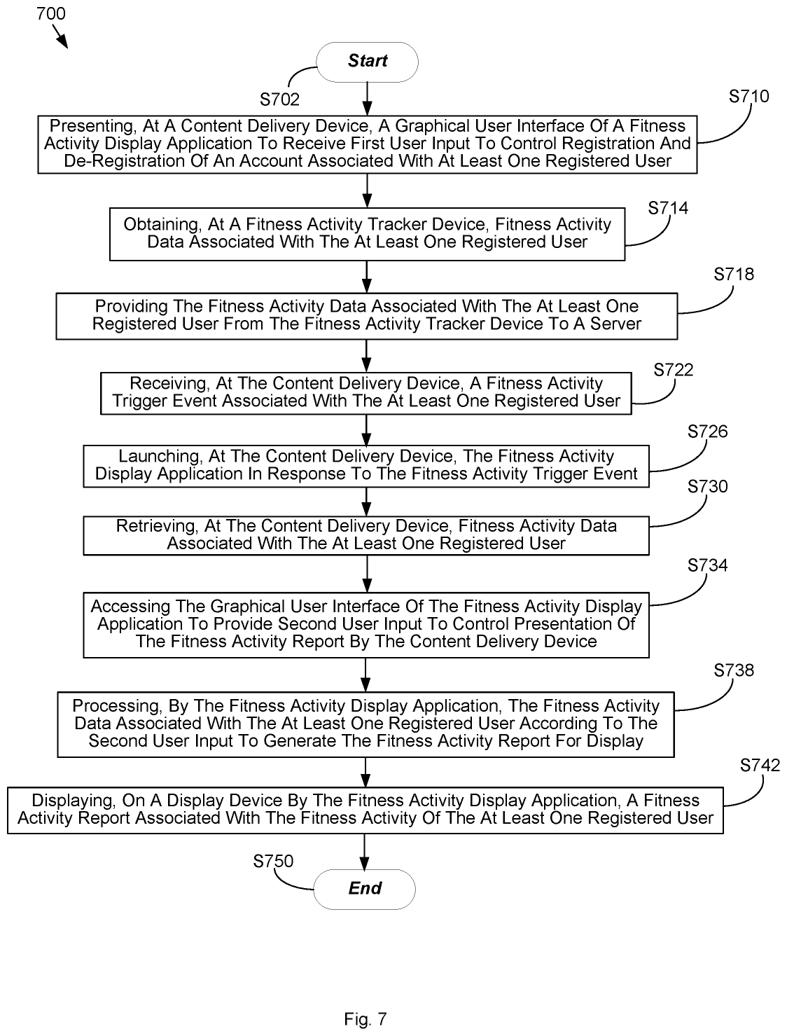

FIG. 7 is a flow chart of a method 700 for displaying fitness activity data on a display device. In FIG. 7, method 500 starts (S702), and a graphical user interface of a fitness activity display application is presented, at a content delivery device, to receive first user input to control registration and de-registration of an account associated with at least one registered user S710. For example, upon receiving the fitness activity trigger event 420, a fitness activity display application 432 is launched on the content delivery device. The fitness activity display application 432 sends fitness activity data 434 to a display device 440. Further, user fitness activity tracker accounts 552 for registered users may be created on a server 550 using the user registration and de-registration controls 568. Each user fitness activity tracker account 552 is linked to one of fitness activity trackers #1 510, #2 513, #n 514, and/or one or more mobile devices 520 for storing fitness activity data associated with a user of activity trackers #1 510, #2 513, #n 514 and/or a mobile device 520. The fitness activity data is accessible by a user to view or modify.

Fitness activity data associated with the at least one registered user is obtained at a fitness activity tracker device S714. For example, the graphical user interface 564 also provides controls for user registration and de-registration 568 so user fitness activity tracker accounts 552 may be created at server 550. The fitness activity display application 562 runs in the background to retrieve fitness activity data periodically from fitness activity trackers of all registered users (directly from fitness activity trackers #1 510, #2 513, #n 514, mobile device 520, or server 550).

The fitness activity data associated with the at least one registered user is provided from the one or more fitness activity tracker devices to a server 718. As described with regards to FIG. 5, user fitness activity tracker accounts 552 for registered users may be created on a server 550 using the user registration and de-registration controls 568. Each user fitness activity tracker account 552 is linked to one of fitness activity trackers #1 510, #2 513, #n 514, and/or one or more mobile devices 520 for storing fitness activity data associated with a user of activity trackers #1 510, #2 513, #n 514 and/or a mobile device 520. The fitness activity data is accessible by a user to view or modify.

A fitness activity trigger event associated with the at least one registered user is received at the content delivery device S722. For example, when a fitness key is pressed on a remote control, a fitness activity trigger event is sent to the content control device 560.

The fitness activity display application is launched at the content delivery device in response to the fitness activity trigger event S726. For example, the fitness activity display application 562 may be linked to the fitness key and launched when the fitness key is pressed.

Fitness activity data associated with the at least one registered user is retrieved by the content delivery device S730. As described with regards to FIG. 5, the fitness activity display application 562 runs in the background to retrieve fitness activity data periodically from fitness activity trackers of all registered users (directly from fitness activity trackers #1 510, #2 513, #n 514, mobile device 520, or server 550). Fitness activity data may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc.

The graphical user interface of the fitness activity display application is accessed to provide second user input to control presentation of the fitness activity report by the content delivery device S734. With regards to FIG. 5, GUI 564 provided by fitness activity display application 562 executed by the content delivery device 560 provides presentation controls 566 to manage how information associated with fitness activity data is displayed.

The fitness activity data associated with the at least one registered user is processed by the fitness activity display application according to the second user input to generate the fitness activity report for display S738. For example, when a user requests for a fitness report by pressing the fitness key on a remote control, the fitness activity display application 562 prepares a consolidated report for all registered user fitness activity tracker accounts 552 to display on the display device 580. The fitness activity display application 562 may also learn about the data to present various comparison, recommendation of exercise or diet, animation, and various other reports to help any individual user or family or friends to plan and achieve fitness goals.

A fitness activity report associated with the fitness activity of the at least one registered user is displayed on a display device by the fitness activity display application S742. The content delivery device 560 creates a fitness activity report based on the received fitness activity data that is presented on display device 580. For example, based on the fitness activity data, emergency alerts may also be generated by the fitness activity display application 562 to provide a warning or other message on the display device 580 in case activity readings are above threshold. Display device 580 presents information associated with the fitness activity data from fitness activity trackers #1 510, #2 513, #n 514 in the foreground on a display of display device 580. The method then ends (S550).

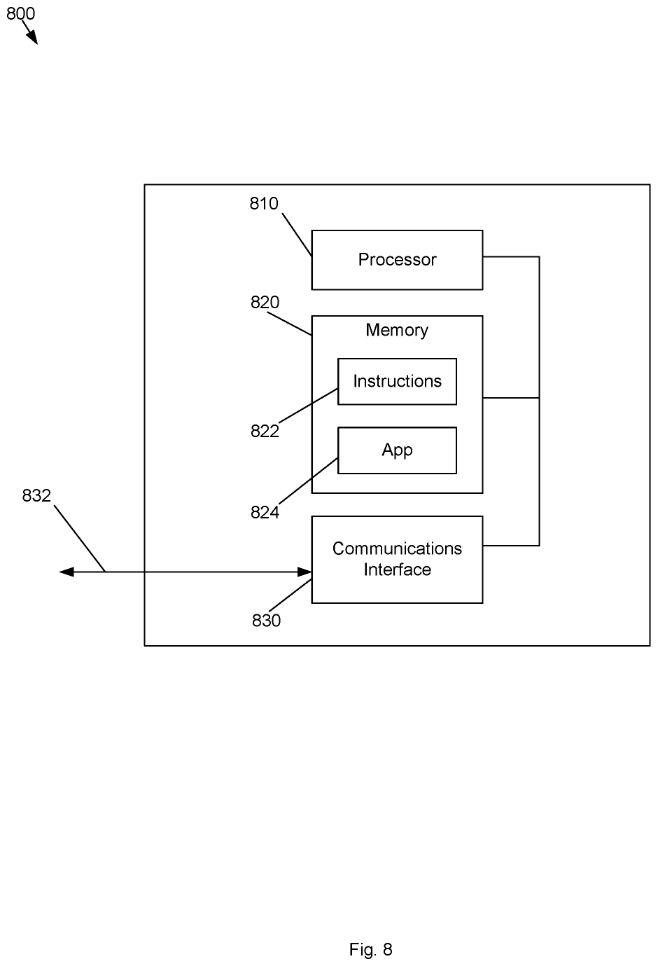

FIG. 8 illustrates a block diagram of a device 800, such as a fitness activity tracker device, mobile device, and/or a content delivery device as discussed above.

In FIG. 8, device 800 includes a processor 810, memory 820, and communication interface 830. Memory 820 may include instructions 822, and an application 824. Communication interface 830 processes data and packets for transmission as well as processing packets that are received by the device 800 via communication channel 832. In device 800, processor 810 may be configured using the instructions 822 in memory 820 to generate an application 824 that is presented at a display device, fitness activity tracker, a content delivery device, or a mobile device. For example, application 824 may be a fitness activity display application that runs in the background on a content delivery device to periodically retrieve fitness activity data from fitness activity trackers of all registered users (directly from fitness activity trackers #1 510, #2 513, #n 514, mobile device 520, or server 550). Fitness activity data may include fitness or health data such as a number of steps taken by a user, a user's heart rate data (e.g., instantaneous or average), sleeping patterns, active time tracking, exercise routines, stress levels, calories used, a current or average blood pressure, a blood glucose measurement, fitness or health suggestions, etc.

The subject matter of the present disclosure may be provided as a computer program product including one or more non-transitory computer-readable storage media having stored thereon instructions (in compressed or uncompressed form) that may be used to program a computer (or other electronic device) to perform processes or methods described herein. The computer-readable storage media may include one or more of an electronic storage medium, a magnetic storage medium, an optical storage medium, a quantum storage medium, or the like. For example, the computer-readable storage media may include, but are not limited to, hard drives, floppy diskettes, optical disks, read-only memories (ROMs), random access memories (RAMs), erasable programmable ROMs (EPROMs), electrically erasable programmable ROMs (EEPROMs), flash memory, magnetic or optical cards, solid-state memory devices, or other types of physical media suitable for storing electronic instructions.

Further, the subject matter of the present disclosure may also be provided as a computer program product including a transitory machine-readable signal (in compressed or uncompressed form). Examples of machine-readable signals, whether modulated using a carrier or unmodulated, include, but are not limited to, signals that a computer system or machine hosting or running a computer program may be configured to access, including signals transferred by one or more networks. For example, a transitory machine-readable signal may comprise transmission of software by the Internet.

Separate instances of these programs can be executed on or distributed across any number of separate computer systems. Thus, although certain steps have been described as being performed by certain devices, software programs, processes, or entities, this need not be the case. A variety of alternative implementations will be understood by those having ordinary skill in the art.

Additionally, those having ordinary skill in the art readily recognize that the techniques described above can be utilized in a variety of devices, environments, and situations. Although the subject matter has been described in language specific to structural features or methodological acts, it is to be understood that the subject matter defined in the appended claims is not necessarily limited to the specific features or acts described. Rather, the specific features and acts are disclosed as exemplary forms of implementing the claims.

What is claimed is:

1. A content output device, comprising:
a memory storing computer-readable instructions; and
a processor configured to execute the computer-readable instructions to:
receive a fitness activity trigger event associated with at least one registered user;
launch a fitness activity display application;
retrieve fitness activity data associated with the at least one registered user; and
display, on a display device, a fitness activity report associated with the fitness activity of the at least one registered user.

2. The content output device of claim 1, wherein the fitness activity data is obtained by one of a fitness activity tracker device and a mobile device of the at least one registered user and the fitness activity data associated with the at least one registered user is provided directly to the fitness activity display application.

3. The content output device of claim 1, wherein the fitness activity data is obtained by a fitness activity tracker device of the at least one registered user and the fitness activity data associated with the at least one registered user is stored in a server.

4. The content output device of claim 3, wherein the fitness activity tracker device of the at least one registered user provides the fitness activity data associated with the at least one registered user to a mobile phone, and wherein the mobile phones sends the fitness activity data associated with the at least one registered user to the server.

5. The content output device of claim 3, wherein the fitness activity tracker device of the at least one registered user provides the fitness activity data associated with the at least one registered user to the server.

6. The content output device of claim 1, wherein the fitness activity trigger event associated with at least one registered user is provided by a remote control device having a designated key that when selected generates the fitness activity trigger event that is received.

7. The content output device of claim 1, wherein the fitness activity display application includes a graphical user interface for receiving user input to control presentation of the fitness activity report, and to control registration and de-registration of an account associated with at least one registered user, wherein the fitness activity display application processes the fitness activity data associated with the at least one registered user according to the user input to generate the fitness activity report for display.

8. The content output device of claim 1, wherein the fitness activity data is associated with two or more registered users, and wherein the fitness activity display application presents one or more of a comprehensive report comparing the fitness activity data of the two or more registered users, a recommendation of at least one of exercise and diet, and fitness goals suggested for the two or more registered users.

9. A method for displaying fitness activity data, comprising:
presenting, at a content delivery device, a graphical user interface of a fitness activity display application to receive first user input to control registration and de-registration of an account associated with at least one registered user;
obtaining, at one of a fitness activity tracker device and a mobile device, fitness activity data associated with the at least one registered user;
providing the fitness activity data associated with the at least one registered user to a server;
receiving, at the content delivery device, a fitness activity trigger event associated with the at least one registered user;
launching, at the content delivery device, the fitness activity display application in response to the fitness activity trigger event;
retrieving, at the content delivery device, fitness activity data associated with the at least one registered user;
accessing the graphical user interface of the fitness activity display application to provide second user input to control presentation of a fitness activity report by the content delivery device;
processing, by the fitness activity display application, the fitness activity data associated with the at least one registered user according to the second user input to generate the fitness activity report for display; and
displaying, on a display device, a fitness activity report associated with the fitness activity of the at least one registered user.

10. The method of claim 9, wherein the displaying the fitness activity report further includes displaying one or more of a comprehensive report comparing the fitness activity data associated with the at least one registered user, a recommendation of at least one of exercise and diet for the at least one registered user, and fitness goals suggested for the at least one registered user.

11. The method of claim 9, wherein the obtaining the fitness activity data associated with the at least one registered user at one of the fitness activity tracker device and a mobile device further comprises obtaining the fitness activity data associated with the at least one registered user at a smart watch of the at least one registered user.

12. The method of claim 9, wherein the providing the fitness activity data associated with the at least one registered user to the server further comprises providing the fitness activity data associated with the at least one registered user from the fitness activity tracker to the mobile device, and sending the fitness activity data associated with the at least one registered user from the mobile device to the server.

13. The method of claim 9, wherein the accessing the graphical user interface of the fitness activity display application at the content delivery device further comprises accessing the graphical user interface of the fitness activity display application at a set-top box.

14. The method of claim 9, wherein the receiving the fitness activity trigger event associated with the at least one registered user at the content delivery device further comprises providing the fitness activity trigger event to the content delivery device by a remote control device having a designated key that when selected generates the fitness activity trigger event received by the content delivery device.

15. A non-transitory computer-readable media having computer-readable instructions stored thereon, which when executed by a processor causes the processor to perform operations comprising:
presenting, at a content delivery device, a graphical user interface of a fitness activity display application to receive first user input to control registration and de-registration of an account associated with at least one registered user;
obtaining, at one of a fitness activity tracker device and a mobile device, fitness activity data associated with the at least one registered user;
providing the fitness activity data associated with the at least one registered to a server;
receiving, at the content delivery device, a fitness activity trigger event associated with the at least one registered user;
launching, at the content delivery device, the fitness activity display application in response to the fitness activity trigger event;
retrieving, at the content delivery device, fitness activity data associated with the at least one registered user;
accessing the graphical user interface of the fitness activity display application to provide second user input to control presentation of a fitness activity report by the content delivery device;
processing, by the fitness activity display application, the fitness activity data associated with the at least one registered user according to the second user input to generate the fitness activity report for display; and
displaying, on a display device, a fitness activity report associated with the fitness activity of the at least one registered user.

16. The non-transitory computer-readable media of claim 15, wherein the displaying the fitness activity report further includes displaying one or more of a comprehensive report comparing the fitness activity data associated with the at least one registered user, a recommendation of at least one of exercise and diet for the at least one registered user, and fitness goals suggested for the at least one registered user.

17. The non-transitory computer-readable media of claim 15, wherein the obtaining the fitness activity data associated with the at least one registered user at one of the fitness activity tracker device and the mobile device further comprises obtaining the fitness activity data associated with the at least one registered user at a smart watch of the at least one registered user.

18. The non-transitory computer-readable media of claim 15, wherein the providing the fitness activity data associated with the at least one registered user to the server further comprises providing the fitness activity data associated with the at least one registered user from the fitness activity tracker to a mobile device, and sending the fitness activity data associated with the at least one registered user from the mobile device to the server.

19. The non-transitory computer-readable media of claim 15, wherein the accessing the graphical user interface of the fitness activity display application at the content delivery device further comprises accessing the graphical user interface of the fitness activity display application at a set-top box.

20. The non-transitory computer-readable media of claim 15, wherein the receiving the fitness activity trigger event associated with the at least one registered user at the content delivery device further comprises providing the fitness activity trigger event to the content delivery device by a remote control device having a designated key that when selected generates the fitness activity trigger event received by the content delivery device.
